(19) United States
(12) Patent Application Publication
(10) Pub. No.: US 2022/0205964 A1
(43) Pub. Date: Jun. 30, 2022

(54) System and Method for a Remotely Deployable, Off-Grid System to Autonomously Detect, Quantify, and Automatically Report Emissions of Methane and Other Gases to the Atmosphere

(71) Applicant: Scientific Aviation, Inc., Boulder, CO (US)

(72) Inventors: Stephen Arthur Conley, Boulder, CO (US); Thomas Bleha Ryerson, Boulder, CO (US)

(21) Appl. No.: 17/133,930

(22) Filed: Dec. 24, 2020

(51) Int. Cl. G01N 33/00; G01M 3/00

(52) U.S. Cl. CPC G01N 33/0036; G08C 17/02; G01M 3/007

(57) ABSTRACT

A system and method for a remotely deployable, off-grid system to autonomously detect, quantify, and automatically wort emissions of methane (CH₄) and other gases to the atmosphere, Automated CH₄ emissions detection is accomplished by the use of commercially available CH₄ sensors. CH₄ accuracy is maximized by simultaneously measuring, and accounting for, undesired CH₄ sensor response from interfering gases such as carbon monoxide (CO) and water vapor (H₂O), and undesired CH₄ sensor response from ambient temperature (T) changes. Automated CH₄ emissions quantification is accomplished by calculating a leak rate (mass or volume per unit time) from the measured concentration enhancements using simultaneous measurements of wind speed and direction. Automated CH emissions reporting is accomplished following transmission of measured CH₄concentrations via cellular wireless, radio, or satellite link to a central cloud-based server. Remote off-grid operation is accomplished by solar, wind, or other renewable energy source(s) that charge an on-board battery. This system offers a robust, unattended, and continuous CH₄ monitoring and reporting capability to permit improved accuracy and efficiency of CH₄ leak detection and repair (LDAR) from sources located in remote areas without electrical power, e.g., leak detection at well pads and processing facilities in oil and gas production areas, at concentrated animal feeding operations, and other methane sources.

### BACKGROUND-PRIOR ART

The following is a tabulation of some prior art that presently appears relevant:

| Pat. No. | Issue Date | Patentee |
|---|---|---|
| None Found | | |

### BACKGROUND OF THE INVENTION

This invention relates generally to a system and method for autonomously detecting, quantifying, and automatically reporting CH₄ leaks to the atmosphere. The invention relates more specifically to a system and method that uploads data autonomously to a central server from one or more CH₄ sensor devices located on the fence line around a CH₄ source, each device consisting of a CH₄ sensor corrected for cross-sensitivities to interfering gases and ambient temperature for improved CH₄ leak detection, quantification, and geolocation and reporting, said system being capable of off-grid operation by means of solar, wind, or some other renewable energy source that charges an on-board battery.

A specific need exists for autonomous and accurate detection, quantification, and automatic reporting of CH₄ emissions (“leaks”) to the atmosphere. CH₄ is flammable, contributes to background ozone pollution, is a potent greenhouse gas, and is a valuable commodity.

Continuous CH₄ monitoring is increasingly needed to reduce the risk of flammable leaks, identify and address sources of pollutant and greenhouse gas emissions, and to reduce saleable product losses. CH₄ emissions to the atmosphere come from a variety of natural and human sources, and national and international policies to identify and reduce these emissions is of increasing priority. Oil and gas production areas are a significant source of CH₄ to the atmosphere, but the location, timing, and magnitude of CH₄ emissions are often poorly quantified. A typical oil and gas production basin can encompass hundreds of square miles, with hundreds of thousands of potential CH₄ emission sources from the tens of thousands of well pad, gathering, and transmission facilities within a typical basin. Similarly, large concentrated animal feeding operations (CAFOs) can consist of tens of thousands of livestock, multiple sewage lagoons, large manure storage piles, and other sources of CH₄ emissions to the atmosphere. Finding and mitigating CH₄ emissions at their source has the potential to reduce economic losses, improve air quality, and minimize the climate impacts of the energy production and agricultural practices needed to power and feed a growing global population. Thus, inexpensive, unattended, and autonomous monitoring systems are required to provide a robust and economically feasible continuous CH₄ emissions monitoring solution suitable for extensive field deployment and operation.

Many commercially available research-grade CH₄ detectors, e.g., the Picarro model G2301 or Los Gatos model 915-0001, are optimized for ambient atmospheric measurements at ultra-trace levels well away from source regions. These detectors offer extremely high sensitivity, high selectivity for CH₄, and high instrument stability over time, but can require skilled operators, typically consume tens to hundreds of watts of AC power, and cost tens of thousands of dollars or more for each detector. These research-grade detectors are cost-prohibitive for use in a continuous emissions monitoring network set up to detect, quantify, and specifically attribute methane leaks to individual sites among thousands of well pads in an oil and gas production region, or at the thousands of CAFOs distributed throughout the agricultural regions of the U.S. A wide range of less precise, lower-cost CH₄ detectors are commercially available, for both personal exposure monitoring (e.g., the Honeywell GasAlert Extreme) and for combustible gas leak detection (e.g., the Bacharach Leakator®). These detectors are less sensitive than the research-grade detectors mentioned previously and are more prone to undesired sensitivities to gases other than CH₄ which can lead to erroneous “false positives,” especially from other combustible gases such as hydrogen, ethanol, and/or carbon monoxide. Typically, these detectors are designed for a fixed installation and require AC power or are designed to be hand-carried and require frequent battery replacement. Detector costs range from hundreds of dollars to thousands of dollars. These CH₄ detectors are not typically designed for long-term unattended use in remote locations without reliable AC power and do not provide telemetry of measured CH₄ values to a cloud-based server. Modern microfabrication technology has enabled a new class of commercially available, miniaturized, and inexpensive CH₄ sensors, typically based on metal oxide semiconductor (MOS) or electrochemical cell (ECC) detection of CH₄. These sensors are compact (˜1cm³ in volume) consume very little power (tens of milliwatts) and are sufficiently inexpensive (˜$10 per unit) to enable cost-effective large-scale deployment. Drawbacks to these MOS-type sensors include significant interferences from non-target gases such as carbon monoxide (CO) and non-methane hydrocarbons (NMHCs) and an undesired sensor response from changes in environmental temperature (T) and water vapor (H₂O). Without the ability to specifically measure and correct the raw sensor output for interfering gases and ambient temperature changes, these MOS or ECC sensors will suffer from false positives, i.e., spurious CH₄ detection due to temperature and humidity changes or due to elevated levels of interfering chemical species. Avoiding false positives from undesired sensitivity to chemical or environmental interferences is essential to maximize the reliability of a CH₄ monitoring system, to enhance cost-effectiveness of its incorporation into a large-scale leak detection and repair system, and to accurately monitor CH₄ leaks from thousands of potential sources in remote areas.

### SUMMARY OF THE. INVENTION

The present. invention is an integrated hardware and software system that consists of one or more pole-mounted detector boxes installed at fixed locations around the perimeter of a facility to be monitored communicating with cloud-based software for data processing and information dissemination. Each detector box can be solar powered for off-grid use and has sufficient on-board battery capacity for several days of operation without charging. GPI coordinates are determined for each detector box at installation, along with coordinates for components of interest (wellheads, tanks, separators, flares, etc.) at the monitored facility. Once powered, the system continuously detects CH₄ using an inexpensive, commercially available metal-oxide semiconductor (MOS) sensor. Ancillary measurements of ambient temperature and humidity are included in each detector box, and measurements of CO are optionally available in each detector box. One box at each monitored facility is equipped with an ultrasonic sensor to measure horizontal wind speed and direction. Chemical sensor voltages, ambient temperature, and relative humidity data are sampled multiple times per second and wind speed and direction data are sampled once per second. 1-minute averages and the standard deviation of the wind direction are calculated, encrypted, and transmitted every 5 minutes by an embedded microprocessor equipped with either a cellular radio or wifi radio to a cloud-based server. Software on the server applies calibrations to compensate for cross-sensitivity to temperature, humidity, and CO to convert CH₄ sensor voltages into CH₄ mixing ratios in parts per million (ppm), which are logged and displayed along with the ancillary data as a time series on a browser-accessible dashboard. Data are encrypted and available for download in various formats by authenticated users. Deriving and displaying CH₄ leak rates, rather than just CH₄ concentrations, is a crucial step that greatly enhances the information provided by a continuous monitoring system, offering a more accurate picture of actual leak size by normalizing the effects of atmospheric dispersion on concentration. The system software automatically incorporates wind speed, wind direction variability, and derived atmospheric stability parameters as input to an atmospheric plume dispersion model (e.g., van Ulden, Atmos. Environ., 1978) to calculate, log and display 15-minute-averaged CH₄ leak rates as a time series plot in the dashboard. Errors in simulating atmospheric dispersion increase during periods of light and variable winds, so CH₄ leak rates are not reported for wind speeds below 1 meter per second and for 15-minute-average wind direction variability in excess of ±30° .

The usefulness of a targeted LDAR program depends critically on the ability of a continuous monitoring system to reliably detect and quantify CH₄ leak rates (not just CH₄ concentrations), geolocate probable sources, and alert operators to any leaks that rise above some actionable threshold for a given facility. In practice this threshold can vary with facility size, operator requirements, applicable regulations, and other practical considerations. The present system alert threshold is fully user-configurable but by default sends automated text or email alerts to operators when a 4-hour running mean of CH₄ leak rates in excess of 2 standard deviations above a 30-day running mean is detected at a monitored facility. Additional calculations use average wind direction and its 15-minute variability to generate an approximate upwind source footprint and automatically identify potential source locations at the monitored facility. Automated alert information transmitted to the operator includes the facility name, location, leak rate and its uncertainty, and the most probable component(s) to which the leak is attributed as a guide for LDAR team response. Leak geolocation accuracy depends on atmospheric transport conditions and improves over time, especially when leak detection by two or more detector boxes permits triangulation to a specific source location or facility component. For each 15-minute average leak rate calculation, the system also generates a .kml file depicting facility components, detector box locations, and the calculated upwind footprint for the leak to provide a visual representation in Google Earth to guide LDAR team decision making (FIG. 3).

### DRAWING-FIGURES

FIG. 1 shows a schematic diagram showing major components (exhaust vent, battery, solar controller, sensor and processor board, and intake fan) within the outer solar-powered detector box enclosure; the version using external AC power lacks the battery and controller. Internal and external wiring and connectors are not shown.

FIG. 2 shows a pole-mounted installation with wind sensor, detector box, and solar panel.

FIG. 3 shows a screen shot from the system dashboard showing a Google Earth view of an instrumented wellpad with locations of eight detector boxes (numbered circles) and facility components (squares). The footprint for each 15-minute period is automatically generated from wind direction and variability data and shown as a shaded triangle upwind of the detector box registering a leak. In this example, the system automatically and correctly identified the southwestern tank (indicated by the arrow) as the most probable leak source.

### DETAILED DESCRIPTION

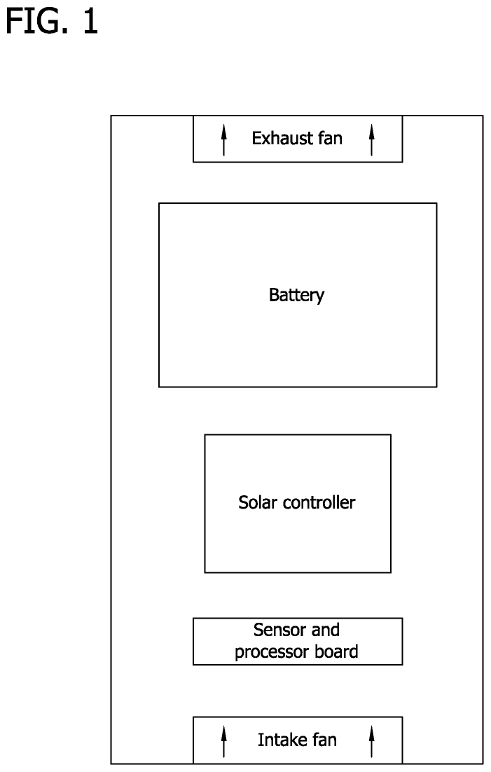

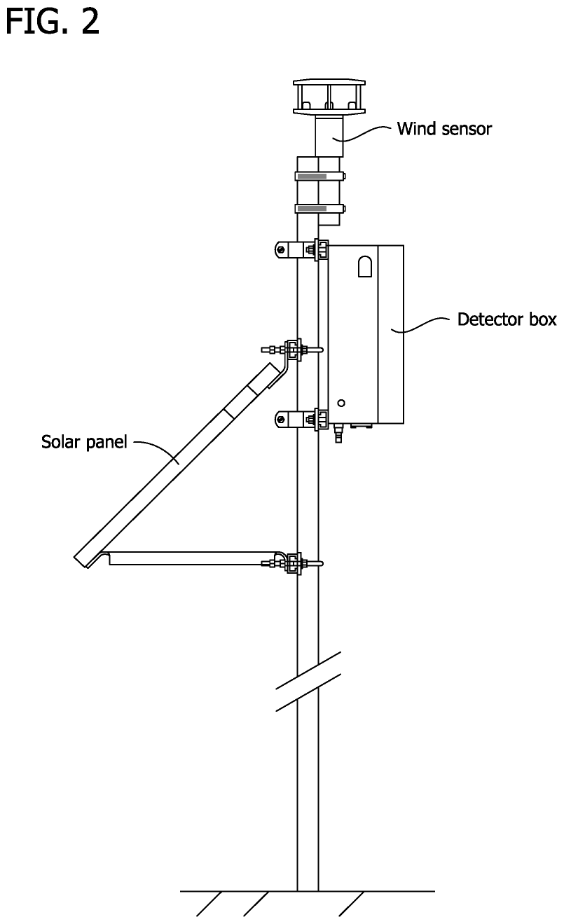

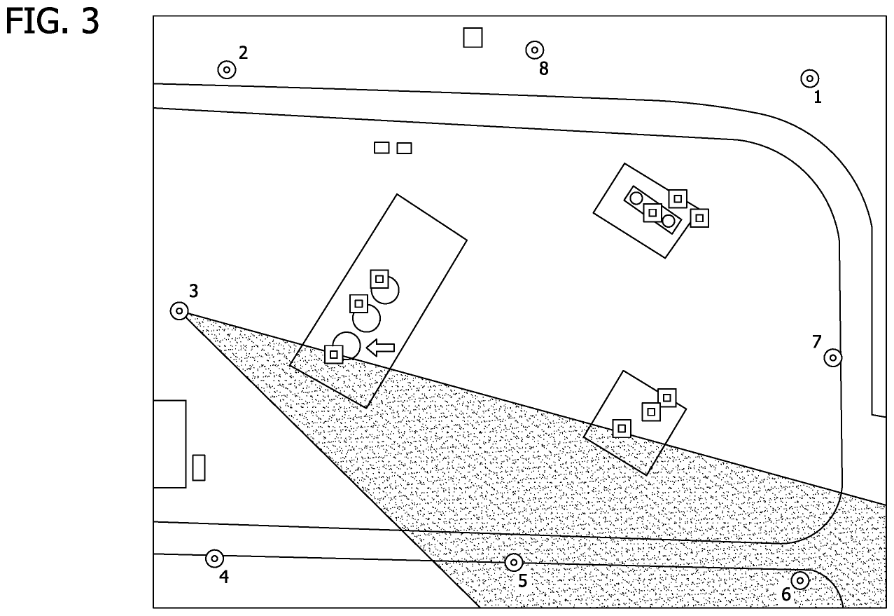

The present invention simultaneously measures ambient temperature (T), ambient relative humidity (RH) and optionally ambient carbon monoxide (CO) and corrects the raw CH₄ sensor signal to account for these confounding factors, maximizing CH₄ accuracy and reduce the incidence of “false positive” leak reports. One or more CH₄ detector boxes (FIG. 1) are installed on poles (FIG. 2) around the perimeter of a monitored facility (FIG. 3) to detect CH₄ leaks at the fence line regardless of the prevailing wind direction. At least one box installed at a given CH₄ source location equipped with a wind speed and direction sensor, typically, a sonic anemometer, to permit a leak to be attributed to that site, and the leak rate estimated from measured CH₄ concentration data using mass balance calculations and a plume diversion model. All detector boxes include an automated remote communication ability via cellular wireless, radio, or satellite link to a central cloud-based server. Detector boxes of the present invention typically accept power from a solar, wind, or other renewable energy source that charges an on-board battery for continuous, unattended, remote, off-grid operation. Optionally, detector boxes can accept grid-tied AC or DC input power where available. Data upload is managed to reduce transmission events and the majority of data processing takes place on the cloud-based server to decrease power consumption by the detector box. Power consumption is minimized throughout the detector box by selecting a low-power fan and microprocessor, leading to an overall continuous power draw of <1.5 W to maximize off-grid uptime for a given renewable power configuration. Once data are transmitted to the cloud, the system calculates 1-minute-average chemical mixing ratios and applies an atmospheric dispersion model to derive 15-minute-average leak rates. These data are archived, displayed as time series plots on the dashboard, and made available for download. For each detector box that registers a leak, the system uses measured wind direction and its variability to calculate an upwind source area (“footprint”) for each 15-minute period and generates a .kml file for visualization in Google Earth (FIG. 3). The system further identities facility components located within the footprint of each detected leak to identify those most likely to be the source. Finally, the system alert threshold is user-configurable but by default sends automated text or email alerts when a 4-hour running mean of CH₄ leak rates in excess of 2 standard deviations above a 30-day running mean is detected at a monitored facility. Automated alert information typically includes the facility name, location, leak rate and its uncertainty, and the most probable component(s) to which the leak is attributed as a guide for LDAR team response.

### CONCLUSION

The ability to remotely deploy and autonomously detect, quantify and report emissions of methane and other gases to the atmosphere is an important step in the evolution of emissions calculation and reduction. This will assist energy producers, regulators, researchers, and other interested parties to better understand the emission profiles of various locations. Unlike more traditional methods that provide an emission profile at a particular point in time and/or provide a concentration level with little or no insight into the emissions profile outside of the particular time of measurement and the actual emission rate, this system will provide a more complete emission profile by capturing emission information on a continuous basis and provide an actual estimated omission rate, including calibrations that correct for factors that can impact the emission calculation such as temperature and relative humidity. The autonomous nature of this invention further allows for the continuous monitoring of facilities to occur without the need to have personnel on-site, allowing increased levels of information related to emissions without the need to increase headcount. This invention will allow a user to automatically learn of a situation at a particular facility that is of interest and/or may require attention in near real-time rather than the more traditional approach whereby emissions may go for days, weeks, or months without being detected or addressed. Although the description above contains many specificities, these should not be construed to limit the scope of the utility of this capability but as merely providing illustrations of some of several uses.

The invention claimed is:

1. A device to selectively and accurately quantify atmospheric CH₄ concentrations, comprised of:
One or more low-power, low-cost CH₄ sensors,
a temperature sensor,
a relative humidity sensor,
a data logger,
a telemetry capability for wireless communication off-site to a cloud-based server,
a renewable power source and batter for unattended, remote, off-grid operation.

2. Optionally, the device as in claim 1, further comprising:
One or more low-power, low-cost sensors for other gases of interest, e.g., hydrogen sulfide (H₂S), and/or potential interferences in the CH₄ measurement, e.g., carbon monoxide (CO), hydrogen (H₂), methanol (CH₃OH), ethanol (CH₃CH₂OH). acetone ((CH₃)₂(CO)), and other hydrocarbons such as ethane (C₂H₆), propane (C₃H₈), isomers of butane (C₄H₁ ) and longer-chain hydrocarbons.

3. The device as in claim 1, further comprising:
a wind speed and direction sensor.

4. Cloud-based software to archive the data, process detector signals, apply calibration data to calculate chemical mixing ratios, and derive leak rates, as well as software that displays raw and processed data as time series, permits data download in various file formats, and prmiuces geolocated results showing probable leak locations and magnitudes, and finally, software that produces automated alerts triggered from calculated leak rates that exceed user-selhtable threshold values.

5. The application of a network of multiple devices and servers in claims 1, 2, 3, and 4 installed to enable fenceline monitoring of gas emissions to the atmosphere, for unattended, automated, off-grid leak detection, quantification, and automatic reporting from a multitude of remote sites.
